(19) United States
(12) Patent Application Publication
(10) Pub. No.: US 2011/0189094 A1
(43) Pub. Date: Aug. 4, 2011

(54) BIOLOGICAL METHOD FOR IN VIVO TRACKING OF MOLECULES AFFECTING CELLULAR MIGRATION

(75) Inventor: Miguel Allende Connelly, Santiago De Chile (CL)

(73) Assignee: UNIVERSIDAD DE CHILE, SANTIAGO DE CHILE (CL)

(21) Appl. No.: 12/990,690

(22) PCT Filed: Apr. 29, 2009
(86) PCT No.: PCT/EP2009/055204

§ 371 Date: Feb. 23, 2011

(30) Foreign Application Priority Data
May 2, 2008 (CL) 1275-2008

(51) Int. Cl. A61K 49/00; C07C 233/10; C07C 47/225; C07C 405/00

(52) U.S. Cl. 424/9.1; 564/123; 568/446; 562/503

(57) ABSTRACT

Cellular migration is a normal part of the normal development of the human being and, in certain pathologies, its alteration damages physiological performance, so that it is necessary to learn about its molecular and cellular foundations and establish methodologies to identify those molecules that modulate this cellular behavior. A screening method using zebra fish embryos is used to follow the advance in the migration of the lateral line primordium upon contact with a test compound. Modulators N found are Fenritinide, PGD2 and is-deoxy-PJE2. The invention refers to a biological method for the IN VIVO, fast, massive and simultaneous tracking of molecules capable of affecting cellular migration, in order to facilitate the for of potential candidates to be used in the diagnosis, prevention, and, principally, for the preparation of pharmaceutical compositions for the treatment of congenital pathologies, immune system dysfunctions, tissue regeneration failure, inflammation, cancer.

### TECHNICAL FIELD

This invention is related to a biological method to track in vivo, rapidly, massively and simultaneously, molecules capable of positively or negatively affecting cellular migration, in such a way that this method considerably facilitates the discovery of potential candidates to be used in the diagnosis, prevention and principally in the formulation of pharmaceutical compositions useful in the treatment of congenital pathologies, immune system dysfunction, tissue regeneration failure, inflammation, cancer, etc. The invention contributes to the discovery of new pharmaceutical alternatives for the treatment of pathologies in superior vertebrates, including animals, and with special preference for the treatment of human diseases. In addition, the molecules identified by the proposed method may constitute powerful tools to elucidate and/or discover molecular mechanisms associated with the control of cellular migration.

### BACKGROUND

In unicellular and multicellular organisms, cellular migration is a fundamental process, involved in embryonic development, in immunity mechanisms and in the regeneration of tissues, among others. On the other hand, the uncontrolled migration of certain cellular types may facilitate, for example, an infection or a cancer expansion. Thus, cellular migration plays a fundamental role, both in the normal state and in the generation and progress of disease stages. Precisely, because of its importance, cellular migration has been concentrating great interest in biomedicine, with the investment of great economic and human efforts in order to understand the molecular and cellular mechanisms that orchestrate cellular migration processes in diverse systems. The understanding of these processes has allowed the detection of molecular systems to be selected as targets in the combat against diseases associated with this cellular phenomenon.

A great number of diseases related to the migration of diverse cellular types has been described. Some examples of these pathological phenomena are: 1) the tumoral cells that decide—for not too clear reasons—to start their circulation, migrate and colonize new tissues, a process known as metastasis; 2) the uncontrolled migration of T lymphocytes towards the lining (synovium) of joints causing heat, swelling and pain in the joint, provoking the autoimmune disease known as rheumatoid arthritis; 3) on other occasions, the T lymphocytes may erroneously go to the beta cells in the pancreas (responsible for the production of insulin), provoking the development of Type 1 Diabetes. In the preceding cases, an effective therapy makes it necessary to inhibit the migration of lymphocytes towards their targets, for example, by means of organ transplants where the individual can be immunosuppressed so that the immune system does not reach the foreign organ and rejects it. Extreme clinical circumstances such as these are some of the main technical problems still unresolved, and, for this very reason, reinforce the need to develop less invasive therapies, hopefully through the simple administration of a new generation of pharmaceutical products. This is precisely the technological contribution of our invention, because it proposes a quick mechanism to track and select new molecules that may be used in pharmaceutical compositions to combat diseases related to defects in the migration of a specific cellular type, independently of the cellular or molecular action level of the molecules detected by the procedure in this invention.

The scientific studies aimed at establishing the relationships among the pathologies associated with the uncontrolled migration of a particular cellular type, have contributed a vast and important information on the molecular factors involved. However, it is still not possible to control the work of these factors and this is one of the objectives of molecular medicine, seeking to prevent suffering in a large population worldwide, affected by pathologies associated with cellular migration failures. These efforts aim at the development of gene therapy and the discovery of natural or synthetic factors that counter the anomalous function of some molecules or stimulate the function of those naturally used by the organism to autonomously combat abnormality cases. A central feature of the invention we propose is to facilitate the discovery of natural or synthetic molecules having a real in vivo capability to modulate cellular migration and, colitis; in the central nervous system, multiple sclerosis and Alzheimer's disease, and others such as rheumatoid arthritis, arteriosclerosis and juvenile diabetes, rheumatism, metastasis, immunodeficiency, etc. in respect of this variety of diseases associated with cellular migration failures, it is necessary to find new cutting edge and high specificity pharmacological compounds.

The advances in the understanding of the molecular mechanisms associated with the migration of cellular groups both in normal conditions and in diseases are described in greater detail below.

Molecular Mechanisms Involved in Diverse Biological Processes Associated with Cellular Migration.

Many prokaryotic and eukaryotic cells have the capability of sensing extracellular signals determining the direction of the source that generates said signal. In this way, they respond changing their shape and moving towards or away from the specific point that originates those high concentrations of the “signal”. This directed migration phenomenon is known as chemotaxis.

Chemotaxis is a cellular response that plays an essential role in embryonic development, in immunity and in adult's tissue homeostasis (Carlos, T. M., 2001). In addition, chemotaxis is key in diverse pathologies related to cellular migration (Condeelis et al, 2005; Moore, MA₁ 2001).

Crucial processes in chemotaxis development are directional perception, polarity and periodic extension of filopodium. In directional perception, the cell detects extracellular signals and generates an amplified internal response with the resulting accumulation of molecules showing the greatest concentration of the external signal (Devreotes and Janetopoulos, 2003; Parent and Devreotes, 1999). Polarization refers to the cell's capability to elongate and define an anteroposterior pattern. Meanwhile, filopodium formation is required for motility and happens spontaneously.

Chemokine receptors (or receptors of chemoattractant molecules) allow several cell types, such as, for example, stem cells, lymphoid progenitors or dendritic cells, to sense gradients of chemotactic cytokines and in this way directionally migrate towards their destinations in the organisms. Cellular migration by means of a gradient of chemokines is accompanied with cell polarization, cytoskeleton arrangements, and adhesive interactions with the extracellular matrix (Sanchez Madrid and del Pozo, 1999). In this context, it has been demonstrated that in leukocytes there are 20 types of chemokine receptors that attach to a G protein designated Gj (Kiinker et al., 1996; Murphy, P. M., 1994) while experiments conducted with neutrophils have demonstrated that the local accumulation of PI-(3, 4, 5)P₃ is responsible for the cells' capability to maintain a directed migration and that this local accumulation of PI-(3, 4, 5)P₃ is regulated by enzymes PI3K and PTEN, suggesting a crucial role in the polarized location of these enzymes, so that the cell may sense properly and migrate in the right direction.

### Cellular Migration and the Immune Answer

In order to obtain an effective immune answer, the interaction of B cells, T cells and dendritic cells {DCs from “Dendritic Cells” in English) is required in the secondary lymphoid organs. Chemokines are important regulators so that lymphocytes may go from the blood stream through the highly specialized endothelial venules (HEVs from “High Endotelial Venules” in English) towards the secondary lymphoid organs, where they will finally initiate an immune response. This is a process developed in multiple steps, with the participation of diverse families of adhesion molecules, such as integrins, selectins and members of the immunoglobulin superfamily (Springer, 1994). In this context, the chemokines appearing in the laminal surface of the endothelium trigger the activation of integrines in the cellular surface of the migratory lymphocytes, leading to a strong adhesion of the lymphocytes to the endothelium, being a prerequisite for the latter to be capable of getting across the endothelium and migrate towards the secondary lymphoid organs. All these diverse factors cooperate jointly to the development and good performance of the immune system, guiding cellular movements in the course of an immune response, and contribute to the lymphoid system's homeostasis.

On the other hand, dendritic cells play a crucial role in the initiation of an immune response. They are designated as the “professional cells” for the presentation of antigens. Immature dendritic cells in the periphery possess a great capability of phagocytizing elements that are foreign to the organism.

Phagocytosis, in association with the stimulation mediated by chemokines at the infection location, leads to the activation of dendritic cells and triggers their maturation. Maturation is characterized by changes in the pattern of expression of chemokine receptors in their surface (Sallusto et al., 1998), reduces the expression of receptors for inflammatory chemokines CCR7 and CXCR4, allowing dendritic cells to migrate towards the secondary lymphoid organs. Here, dendritic cells “present” the information to the 1-naive lymphocytes and an immune response is generated, in addition, CCR7 and CXCR4 contribute to the directed migration of T and B lymphocytes towards the secondary lymphoid organs, and also CXCR4 and its SDF1 (CXCL12) üg and play en essential role in the directed migration of stem cells (Peled et al., 1999); Petit et al. 2002), leukocytes (Aiuti et al. 1997) (Bleul et al. 1996), neurons (Zou et al. 1998) and carcinogenic cells (Muller et al. 2001).

### Cellular Migration and the Immune System in Inflammation

Numerous cellular functions and processes where cellular migration plays a primary role depend on the dynamic changes in the cytoskeleton, among them, the morphogenetic movements during embryonic development, chemotactic movements of the immune system cells, and cohesive migration. Any failure that prevents a proper migration of the cellular components in a system may provoke the development of a pathology.

The leukocyte migration field has created new and prominent clinical opportunities. The molecular signals involved define the migratory control of diverse subgroups of immunitary cells towards or from specific tissues. Since the accumulation of leukocytes in tissues contributes to a vast variety of illnesses, the “molecular codes” that define the directed migration are great candidates for therapy against tissue-specific inflammation. Examples of these illnesses associated with tissue-specific inflammation are inflammatory diseases affecting the skin (Psoriasis and eczemas), intestines (Crohn's disease and colitis), central nervous system (multiple sclerosis and Alzheimer's disease) and others such as rheumatoid arthritis, arteriosclerosis and juvenile diabetes. In this way, specific pharmacological inhibitors for certain leukocytes and their migratory destination may be highly effective in the control of the abovementioned autoimmune diseases. The “anti-migration” therapy interferes with the pathological attraction of these cells. Some diseases may be treated blocking a migration signal that is essential to the recruitment of pathologic cellular subgroups.

Among the proteins involved in migration, there are adhesion molecules such as the E and P-selectins, which are expressed in inflamed endothelial cells and are important in the recruitment of neutrophils, monocytes, T lymphocytes and immature dendritic cells.

Precisely, dendritic cells have a crucial role in the adaptive immune response. These cells instruct T cells to respond against exogenous antigens. Dendritic cells collect and process the exogenous material and, in response to maturation signals, migrate towards the secondary lymphoid organs to initiate the activation of T lymphocytes. Failures to recognize what properly belongs from what is foreign may trigger autoimmune reactions. Therefore, drugs that interfere with the maturation of dendritic cells and their subsequent migration towards the secondary lymphoid organs may be very effective to block autoimmunity. On the other hand, just like lymphocyte migration may be undesirable from the clinical point of view, it also plays a most important role in the endogenous response to tumors. This is how dendritic cells may detect cancer cells and initiate their destruction by the immune system. Tumors evade this defense system producing molecules that inhibit the migration of dendritic cells, becoming invisible to the immune system and allowing tumoral progress. Therefore, molecules that facilitate the immune migration or inhibit the anti-migration mechanisms of cancer may be effective in the development of anti-tumoral drugs.

On the other hand, chemoattractant molecules (chemokines) and their GPCRs (from “G Protein-Couple Receptors” in English) receptors constitute the most diverse group of molecules that define tissue-specific cellular traffic. Their molecular diversity and selective action in diverse leukocyte subgroups, in addition to their restrictive temporal and spatial expression pattern, provide key mechanisms for proper operation of the immune response Inhibitors of the GPCRs and/or their ligands, or their expression in affected tissues, are promissory targets to attenuate cellular migration in diverse type of diseases related to inflammation. Studies in animal models have demonstrated that drugs that inhibit the chemo attractive route may generate powerful anti-inflammatory activities. At present, clinical studies are being conducted to determine their effectiveness in specific human diseases.

Specific combinations among GPCRs and their ligands may activate the integrine-dependent adhesion (adhesion molecules) of leukocytes to the inflammation sites. Next, molecules of the Rac family of GTPases and protein kinases such as PI3K are activated, which promote the mobility of leukocytes to cross the endothelial barrier. The gamma isoform of P13K is important in the directed migration of macrophages and dendritic cells. There are diverse P13K isoforms, which define different migratory functions in diverse cellular groups, in this way, the PI3K kinase family constitutes an important therapeutical target in a variety of inflammatory diseases.

### Cellular Migration and Cancer

At the present time, little is known about the routes of the signals that regulate the progression of a tumor and the acquisition of the metastatic phenotype. In addition to the uncontrolled proliferation and cellular death control, the transition to a metastatic-invasive stage with an increased migratory, invasive and disseminative capability, represent critical steps in tumoral progression. The number of signal cascades known to be essential in early embryogenesis and to induce tumorgenesis and promote tumoral invasion and metastasis, increases. The exit of tumoral cells from the epithelium is characterized by dramatic changes in cellular form and function. The entire process is denominated epithelium-mesenchyme (EMT from “epithelial-mesenchymal transition” in English). In addition to the loss of cell-cell contact, processes such as the rearrangement of the cytoskeleton, cellular polarization, cellular orientation or dynamic reorganization of the extracellular cell-matrix contacts, are carried out to induce motility and directed cellular migration across long distances in the organism.

An intense investigation of chemokines led to discover that these chemoattractant molecules and their receptors play a leading role in tumoral invasiveness (Muller et al. 2001; Robledo et al. 2001; Taichman et al. 2002; Zeelenberg et al. 2003). For example, the CXCR4 chemokine is expressed in several cancer types and its SDF1 (CXCL12) ligand is expressed at high levels in lungs, liver, bone marrow and lymphatic nodes, tissues that represent common metastatic destinations for many carcinogenic types. Data obtained using the mouse model demonstrated the role of chemokine receptors in a cancer in progression and in metastasis in the liver.

This is how the factors that control cellular migration or the signal cascades that generate movement, presently constitute selection targets for antkumoral therapy, tissue regeneration, autoimmune diseases, transplants and therapies with stem cells.

### BRIEF SUMMARY

As described above, many pathological processes are generated or projected through the uncontrolled migration of certain cellular types, where this lack of control is caused by defects in the molecular activities of many different factors and also at diverse cellular levels (nuclear, cytoplasmic, cellular membrane) and extracellular levels (extracellular signaling factors, extracellular matrix). Even more so, the state of the art identifies several genie products directly involved in the control of general migration, for example, chemokines, cellular receptors, proteins associated with the cytoskeleton, adhesion molecules, kinases, transcription factors, etc. Each one of these factors is considered as a potential target to control the associated pathologies. However, the discovery of molecules that allow the control of these cellular factors and thereby therapeutically modulate cellular migration is a field that advances slowly. Principally because the search of pharmaceutically useful modulators generally implies focusing on a single cellular factor as the modulation target. In addition, it requires an initial in vitro study that does not always gives the same results when applied to an in vivo organism, which translates into a great difficulty in finding new really useful drugs in the effective treatment of these pathologies. The preceding becomes evident with the fact that most therapies now used against pathologies associated with cellular migration are merely palliative. The state of the art does not have, up until the development of this invention, a robust methodology for massive molecule tracking that shortens the times needed to find molecules with therapeutical potential and that, concurrently, selects these molecules from the beginning on the basis of their therapeutical effect in the most real scenario: in vivo and in toto.

Precisely, our invention proposes a biological system—in vivo and in toto—for the multiple tracking of hundreds of molecules, allowing the selection of those capable of affecting cellular migration, in order to facilitate the finding of potential candidates to be used in the diagnosis, prevention and treatment of diseases associated with cell migration (Ex.: uncontrolled immune system, cancer and metastasis, tissue regeneration, etc.). The invention is based on the search of molecules that affect cellular migration as a global phenomenon, without the need to focus on specifically modulating one of the many of the known molecules participating in this cellular process. The scope of the proposed invention is applicable, in general, to the finding of drugs to be used in the control of cellular migration processes in superior vertebrates and even more preferably for their application in human medicine. In addition, and this will be understood by the individual who is knowledgeable of the art, the proposed procedure will allow the finding of new molecules, whose subsequent analysis may even lead to the discovery of new factors as well as new mechanisms associated with migration control. In other words, this is a ample discovery method, useful to detect anti-migratory or pro-migratory activity of molecules, extracts or compounds of diverse origin.

### DETAILED DESCRIPTION

During the last decade, the zebra fish (Danio rerio) has been used as one of the best models to study vertebrates' development. Its similarity in respect of signal routes, development processes, gene conservation, among others, makes it a trustworthy model. This fish offers many advantages as a discovery tool. Among them we find large scale mutagenesis; its small size, making it possible to keep hundreds of lines in limited spaces; its high fecundity (obtention of close to 200 embryos per mating); it is transparent, allowing a better visualization of its internal development; its fertilization is external, allowing their easy manipulation; its genome is completely sequenced, allowing an easy transgenesis and a great opportunity for the inactivation and ectopic overexpression of genes. In addition, and fundamental for the development of this invention, it has been scientifically confirmed that the zebra fish conserves many of the molecules and functions observed in all vertebrates, including man.

Therefore, a well characterized system or phenotype in a zebra fish provides the additional advantage of facilitating the explanation of the same situation in superior vertebrates, humans included.

### Zebra Fish and the Lateral Line System

The lateral line (LL) is a mechanosensory system found in fish and amphibians, whose function is to detect changes in water pressure and to support behaviors such as the detection of predators, shoal swimming, etc. (Stone, 1933; 1937). The LL is composed of sensory organs known as neuromasts, superficially distributed along the fish body. The LL system is divided into the anterior lateral line system (LLA), with neuromasts in the head (whose lymph node connecting it to the posterior brain is found between the ear and the eye), and the posterior lateral line system (LLP), with neuromasts in the trunk and tail (whose lymph node is found behind the ear). Upon completion of the embryogenesis the LLP is composed of 7 or 8 neuromasts regularly separated and aligned across the horizontal myosept (Metcalfe et al, 1985; Metcalfe, 1989).

Each neuromast is composed of support cells that encircle a group of ciliated cells, identical to the ciliated cells found in the internal ear (Fritzsch, 1988). The neuromasts are generally exposed to the medium at the body's surface, although they are sometimes located in subepidermal channels, and may be easily visualized with Nomarski microscopy or incubating fish with the vital fluorescence marker, 2-Di-4-Asp, DiAsp, or with FM 1-43 (Coliazo et al, 1994). These compounds accumulate in active ciliated cells. Neuromasts have sensorial neurons as nerves, and efferent fibers that control the system's sensitivity. The sensorial neurons are bipolar and extend a central projection towards the posterior brain.

Neuromasts in the posterior lateral line are deposited by the primordium in the posterior lateral line (pLLP) consisting of a group of about 120 cells that migrate across the horizontal myosept, having an approximate width of 4-5 cells and an approximate length of 20-25 cells (Gompel et al., 2001). The pLLP originates in the cephalic placode, which is formed dose to the posterior brain, and behind the otitic placode. In the zebra fish, the primordium starts to migrate caudally across the horizontal myosept about 20-22 hours after fertilization (hpf) (Metcalfe, 1985; Kimmel et al., 1995). During migration, the primordium deposits groups of 7-8 cells at faithfully reproducible intervals, denominated pro-neuromasts that will subsequently differentiate into functional neuromasts. The pLLP concludes close to 42 hpf, and the last deposited neuromast completely differentiates 6 hours later, completing the embryonic pattern of the primary posterior lateral line.

In brief, in a few hours, the group of cells constituting the LLP go from being an epithelium, delaminate, become migratory, and spatially order themselves to form a coherent sensory organ. Since the behavior of these cells is homologous to the behavior of cells that migrate in an orderly way in other biological systems, in addition to the fact that there is a high molecular conservation among the diverse vertebrate species, we propose that the lateral line is a simplified and accessible biological system, both for the study the cellular migration phenomenon and for the discovery of factors that disturb it, and where the results obtained using this system are applicable to the vertebrate level in general.

Previously, Sapede (2003), has suggested the use of LLP from zebra fish to study metastasis. However, the author makes exclusive reference to the use of LLP in the study of molecules that may antagonize the CXCR4-ligand SDF 1 receptor system involved in the directionality in the movement of LLP cells. The proposal of Sapede (2003) is related to the study of only one type of factors (antagonists) that can control just one (CXCR4-ligand SDF 1 receptor) from the many systems involved in metastasis, which, in turn, is only one among the numerous pathologies associated with cellular migration.

Our invention corresponds to an in vivo massive tracking system to find new molecules, whether natural, synthetic or recombinant, which are capable to positively or negatively affecting any of the mechanisms associated with cellular migration and involved in diverse pathologies.

The molecules selected by this procedure may affect key processes in migration control such as, for example, the cellular motor system, but not only affecting it at the direction control level, but, what is even more important, the molecules selected by the procedure in our invention may act at the level of any of the diverse coupling mechanisms associated with the restructuring of the cytoskeleton during migration (cellular polarization, cellular traction and adhesive cell-cell and cell with extracellular matrix interactions). Thus, our invention is not limited to the exclusive study of factors that affect just one of the multiple mechanisms involved in cellular migration or circumscribed to the effect on only one pair of biomolecules in particular. As already mentioned, the direct participation of diverse polypeptides in cellular migration control has been demonstrated at the molecular level, among them chemokines (for example the bifunctional CXCR4 and SDF1 system), G protein, kinase proteins (ex., PI3K), integrins, cadherines, selectins, GTPases (Rac1, RhoA, RhoB), factors associated with the control of the expression of genes involved in migration, other extracellular ligands (members of the Wnt, FGF, PDGF families), etc. Our invention allows the finding of molecules that may regulate migration at any cellular or extracellular level.

Thus, the procedure we propose, in addition to allowing the finding of new molecules that affect cellular displacement capability or new molecules involved in movement speed, or both, resolves a technical problem consisting of the pre-clinical selection of potential molecules that contribute to combat different pathologies associated with cellular migration such as metastasis, inflammation, lymphocyte homing, Psoriasis, eczemas, Crohn's disease and colitis, multiple sclerosis and Alzheimer's disease, rheumatoid arthritis, arteriosclerosis and juvenile diabetes, arteriosclerosis, rheumatism, metastasis, immunodeficiency, etc.

In addition, this invention facilitates the systematic analysis of hundreds of molecules in one test, satisfying current pharmaceutical demand for systems known worldwide as “high throughput” or high effectiveness systems.

### Detection Method

The innovation we propose, associated with the use of the LLP as a discovery tool, consists in our conceptualization of an effective alternative to detect changes in cellular behavior in a multifactor, robust, consistent, fast, simple and economical way. We have designed a protocol that will allow the detection of agents that modify the behavior of LLP migratory cells with no intervention other than exposing—in vivo and in toto—the LLP cells to the agent. Even more so, the same analysis will determine whether the effect of the agent incubated in the fish occurs in respect of the migration per se, or on cellular adhesion, or at the movement direction level and/or the organization of the migratory cells set. The type of molecules or substances that the method intends to detect may be those that act externally on the fish (agents added to the water or the incubation medium) or internally {proteins expressed by the migratory cells themselves). The exposure will be made while the primordium is migrating actively, that is, in zebra fish larvae from 22 to 36 hours post fertilization (hpf). The key to the effective detection of the modifying activity of cellular activity in the LLP primordium is the existence of a phenotype that can be easily evaluated. One analysis method is to examine the formation or non-formation of a functional system after each treatment, for example, through the tinction of functional neuromasts with DiAsp or FM 1-43. This is a very good primary tracking, to quickly discard those treatments having no effect on system development, cellular migration included. However, the mere absence of mark after incubation with vital tinctions (DiAsp, FM1-43) is not a synonym of migratory problems, but may signal effects on other aspects of normal system development (for example, differentiation of the ciliated cells). In order to examine the direct effects on migration, multiple methodologies are available. Tinctions that reveal the expression of genes {in situ hybridization) or proteins (immunodetection) are possible given the existence of probes or antibodies suitable for this purpose. However, these detections are somewhat laborious and require extended treatments of animals and a detailed microscopic observation. In addition, it is necessary to fix the fish in a determined stage and compare the primordium position in control animals. Our proposal is based on the option of making the observations in living animals, with no intervention or manipulation other than, perhaps, applying a mild anesthetic to immobilize the larvae at observation time. We propose two options for this analysis. First, it is possible to incubate the larvae with vital tinctions that highlight the body's surface cells and these may be observed under fluorescent lighting with an appropriate dissection loupe. One of these compounds is Bodipy (Molecular Probes), which is added to the water marking skin cells and, in a very obvious way, to the migratory primordium of the LLP. The second way to observe these cells during their migration is by means of the specific expression of the Green Fluorescent Protein (GFP). The expression of this protein in transgenic animals allows the observation of living cells clearly distinguishable from the surrounding cells (that do not express the protein) thanks to the emission of green fluorescence when they are illuminated with a light having the appropriate wavelength (Chalfie and Sulston, 1981; U.S. Pat. No. 5,491,084). Therefore, a transgenic fish carrying adjustable DNA that directs the expression in migratory primordium in association with codifying DNA for GFP, would meet the requisite of in vivo visualization of this group of cells. The use of these transgenics is known in the state of the art (Gilmour et al., 2004) and others have been successfully used in our laboratory (Sarrazin et al., 2006) (Hernandez et al. 2007).

With relation to the mechanism to expose fish (larvae) to the agents to be tested, this would be done in two ways. In the first one, agents are directly added to the water or larvae incubation medium in the diverse concentrations to be tested. The volumes must be small, 1-2 ml so that microcups deposited on plastic plates can be used, where up to four 28 hpf larvae are placed. The exposition of larvae will be for a minimum 6-hour period, and during this time the primordium in untreated wild embryos should move along the larva's trunk towards the terminal position in the vitelum extension (next to the anus). Depending on the characteristics of the agent to be tested, we may add to the medium compounds or solvents to help their dispersion. For example, hydrophobic molecules may be firstly dissolved in ethanol before they are added to the water. Most of the molecules or agents to be tested will be added in association with DMSO (di-methyl sulfoxide) to assist the molecules' solubility and availability to the cells in the larvae. In each test, control embryos are incubated in parallel in pure a pure medium or only having adjuvants (ethanol, DMSO, etc.). in order to visualize the larvae, it is not necessary to remove them from the cups but they are directly observed in the fluorescence loupe, adding an anesthetic that immobilizes the larvae making it possible to rapidly evaluate the position of the migratory primordium and, in this way, select among the molecules under analysis.

We have observed that not all the molecules added to the water can penetrate the epidermis of the larvae and produce some effect on the migratory primordium's cells. This is particularly important in the case of large molecules such as proteins. This is why we propose the second application of this methodology. It consists in expressing the molecules (proteins) to be tested in the same fish, in all the cells in the organism, at the moment when the primordium migration is taking place. This may be achieved in diverse ways but we propose one that is the most robust in current use for the heterologous expression in zebra fish. The activity on the primordium migration of the diverse proteins to be tested would be found through the cloning of the respective genes for those proteins in an expression vector (plasmid or transposon that contains a promoter, the gene to be tested, and a transcription termination sequence). In this way, the injection of fish with the DNA (expression vector) in the stadium of a cell, would achieve the incorporation of this DNA in the fish genome and its subsequent expression. A difficulty that might show up is the perturbation in the embryonic development of the fish when they express the protein to be tested. This is relevant because many proteins have multiple functions during development, frequently in diverse tissues, and, therefore, their ectopic expression might generate highly pleiotropic phenotypes that would make it difficult to interpret the phenotypes in the lateral line. Consequently, we propose the use of expression vectors that allow the induction of the expression. In this sense, a useful example is the utilization of temperature-inducible promoters (heat shock), the most known among them in the zebra fish is the hspïO promoter. This promoter has been repeatedly used in this animal with robust results (Halioran et al., 2000). In order to obtain the induction at the right time in the case of this proposal, it would be necessary to raise the embryos' temperature to 37 degrees Celsius during 30 minutes, at approximately 20 hpf, so as to provide time to the induced gene to express itself as functional protein. The phenotypic analysis would be performed as described above.

The use of larvae distributed in microcups on plates not only facilitates the manipulation of a large number of animals in a limited space, but it also allows the simultaneous dispensation of agents, in multiple cups with micropipettes having 8 or 12 points, representing a great technical advantage that our invention is contributing to the state of the art.

What are the types of molecules that our invention pretends to discover? The finding of chemical agents that perturb the migration of the lateral line primordium of the zebra fish may affect fundamental processes in the migration of any cellular type, not only in the fish. Since many of the important molecules in vertebrate animals' biology are conserved, there is a great expectation that said compounds have similar effects on any cell where those same mechanisms are in operation. This would include cells in the nervous, immune or vascular system, in addition to tumoral cells of diverse origin. The same would be valid for the application designed to detect proteins (secreted, cellular membrane or intracellular) having this effect. Evidently, our invention is tool to discover molecules (artificial, natural or recombinant) that may potentially have a universal effect on migratory cells' systems. The power of this tool is based on the precept that, in Nature, there is a finite number of biological systems involved in cellular migration, where these systems are conserved at least among vertebrates. The application of our invention consists in delivering as a result a set of molecules that are candidates for further testing in other systems. The usefulness of our invention is based on the circumstance that no efficient technical procedure exists today to conduct this kind of massive searches, which not only contributes to bring down test times but also selects candidate molecules already tested in a real scenario: in vivo and in toto. The use of cells in culture, for example, the only existing alternative, is cumbersome, inefficient, nonspecific, costly and imprecise, also being biologically irrelevant because it is conducted outside the organism's context.

### Exemplification of the Method

In the following paragraphs we describe three experiments conducted by us that demonstrate the usefulness of the system. These examples only pretend to illustrate the invention white not limiting it, because the individual knowledgeable of the state of the art will see that it is possible to extend the scope of its usefulness. The following paragraphs demonstrate the potential for the discovery of new molecular activities related to the migration phenomenon.

### DESCRIPTION OF THE FIGURES

FIG. 1 shows how the exposition of transgenic larvae of the zebra fish to 4-HPR generates a delay in the migration of the lateral line primordium (see example 1). The fish were incubated under the described conditions with this agent and we subsequently observed the fluorescence generated by the expression of GFP, regulated by the promoter of ClaudinB in the primordium (arrows). As controls we use DMSO, 9-cis retinaldehyde (9cRA) and all-trans (atRA), and in all of them we observe normal migration. Please note the lag in the primordium position (indicated with an arrow) in fish treated with 4-HPR if we compare it with the migration start point (asterisk).

### EXAMPLE 1

The basic experimental conditions used in the three migration test experiments are:

Zebra fish (Danio rerio) are used, kept in a 14/10-hour light/darkness cycle. Embryos are recovered after natural fish mating and they grow in Petri plates at 28° C. in E3 medium (5 mM NaCl, 0.17 mM KCl, 0.33 mM CaCl₂, 0.33 mM MgSO₄, and 0.1% Methylene Blue) until the reach the development stadium suitable for the test, which is determined according to Kimmel et al., (1995). The larval stadiums are designated in post fertilization hours (hpf) or post fertilization days (dpf).

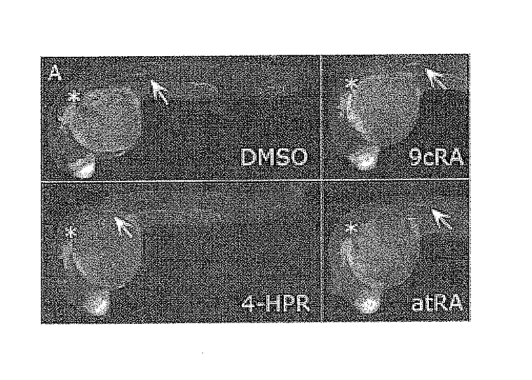

In this first example, in each cup on a plate with 24 cups with 2 ml of E3 medium each, we placed from 3-10 transgenic embryos that express the GFP gene under the control of the CldnB promoter (CldnB::GFP; kindly donated by Dr. Darren Gilmour (EMBL, Heidelberg, Germany). The agents subjected to tests (N˜(4˜hydroxyphenyl)retinamide (Fenretinide, 4-HPR), retinaldehide 9-cis, retinaldehide all-trans and only with the control vehicle: DMSO) were added to the medium at a final concentration of 20 μM when the embryos reached the 22 hpf stadium and the fish were kept in the medium plus the agent until 36 hpf. The GFP mark in the migratory primordium was observed every 4-6 hours in order to monitor its progress. At 36 hpf, we fixed one half of the fish (5 from each cup) with 4% paraformaldehyde dissolved in PBS. We observe the fluorescence of GFP (indicating the primordium position) and evaluated whether it migrated properly or not (FIG. 1). The result shows that it is possible to detect anti-migratory activity in molecules added to the embryos' culture medium using transgenic fish that mark the migratory primordium with fluorescent protein.

### EXAMPLE 2

In this example we pretend to demonstrate how our invention allows extrapolation of the results with zebra fish to superior vertebrates, human beings among them. As we already mentioned, several among the molecules that guide the lateral line primordium migration, as well as the metastatic cells', are common to humans and zebra fish (for example, cxcr4, sdf1, tacstd). Even more important, the migration process is very similar, because in both systems the directed migration depends on the PI3K enzyme (Dumstrei et al., 2004), there is a cytoskeleton rearrangement and the formation of fitopodia and lamelopodia, etc. On the basis of this information, we present below examples that allow us to validate and extrapolate the lateral line system to study and find new immunosuppressant molecules capable of inhibiting cellular migration.

Dendritic cells, when entering the maturation process, start to express chemokine receptors (CCR7, CXCR4) that direct their migration towards the secondary lymphoid organs. We have recently discovered that Fenritinide {4-HPR), a molecule utilized in “clinical trials” against breast cancer and neuroblastoma, inhibits the expression of CCR7 and CXCR4 in dendritic cells in maturation {Villablanca, Allende and collaborators; manuscript in preparation). The inhibition is specific to the chemokine receptors because it does not inhibit other molecules also important for the correct function of the dendritic cell (CD80, CD83, CD86). In this way, Fenritinide would produce a blocking of the mature dendritic cells in the periphery, preventing their migration and arrival at the secondary lymphoid organs, affecting in this way the development of an immune response to pathogens or towards the tumoral cells proper. As negative control we have used the retinoic acid natural retinoid that is not capable of inhibiting CXCR4 in dendritic cells so that it is not capable of inhibiting the migration of these cells.

In this way, we have evaluated our hypothesis of the Fenritinide effect on dendritic cells, taking our invention as the test model. Zebra fish embryos were incubated in accordance with the basic conditions described in Example 1, but in the presence of Fenritinide since the time when the posterior lateral line primordium starts to migrate (22 hours post fecundation (hpf) stadium), until the LLP system is mature (36 hpf stadium) and is easily visualized with the DiAsp tinction. After incubation, the controls (incubated in normal medium or supplemented with retinoic acid) presented mature neuromasts, while the embryos exposed to Fenritinide showed to be negative for the DiAsp tinction. We checked whether the absence of mature neuromasts is caused by migration blocking by means of in situ hybridization using the claudin-B (anti-claudinB antibody, detected with immunofluorescence) primordium marker. The control embryos showed a normal primordium migration and pre-neuromast deposit, while the embryos exposed to Fenritinide displayed a blocking of the migratory primordium at the beginning of its migration, demonstrating that Fenritinide blocks primordium migration, as opposed to retinoic acid having no effect at all. This second example demonstrates a second methodology to detect the anti-migratory activity of the problem compound using simple vital tinctions (DiAsp).

### EXAMPLE 3

In order to confirm that the LLP system is useful to detect molecules that inhibit cellular migration, embryos were incubated as described in the preceding examples, but this time in the presence of prostaglandins PGD2 and 15-deoxy-PJE2, known because they inhibit migration through CXCR4 in human dendritic cells (Nencioni et al. 2002) and PGE2, which increments the expression of CXCR4 in dendritic cells (Scandelia et al. 2002). Embryos in the 22 hpf stadium were incubated between 26 and 28° C., in the absence or presence of either PGD2 or 15-deoxy-PJE2, permitting the development of embryos up to the 36 hpf stadium, at which time we checked the primordium advance both in the treated embryos and in the (untreated) control embryos. Both in the control embryos and in the fish incubated with PGE2, the neuromasts were dyed with DiAsp and we observed that the LLP primordium migrated normally. The embryos treated both with 10 μM PGD2 and 5 μM of 15-deoxy-JE2 were negative for DiAsp and the LLP primordium migration was considerably slower (Viilablanca, Allende and collaborators, unpublished data).

These examples demonstrate how the proposed test is robust and easy to implement following the method herein described.

1. A procedure to select in vivo molecules potentially useful in pharmaceutical compositions to treat diverse pathologies of interest to humans and animals in general characterized because it consists in rapidly and simultaneously tracking several different molecules, which may be natural, synthetic or recombinant and where the method comprises the following steps: a) each molecule to be tested is placed in contact with at least three living embryos of the zebra fish (Danio rerio) at the 22 hours after fecundation stage (hpf) and the embryos are allowed to develop between 26 and 28° C. for 14 hours until the 36 hpf stage. b) the advance in the migration of the lateral line primordium in the treated fish is compared with the primordium migration in control fish that have not been exposed to any contact with the molecule to be tested. c) the following step is to select those molecules that—compared to the control embryos—have significantly affected the primordium migration in the test embryos, be it preventing, delaying, accelerating or making their movement erratic and where these differences in treated fish primordium migration, compared to the control fish, indicate that the tested molecule has an activity with potential use in the treatment of diseases associated with cellular migration.

2. Procedure pursuant to claim 1 characterized because it is feasible to simultaneously track hundreds of different molecules.

3. Procedure pursuant to claim 1 characterized because the primordium migration progress is analyzed incubating the embryos with vital stains allowing the observation of the migratory primordium under fluorescent lighting and using a dissection loupe.

4. Procedure pursuant to claim 3 characterized because Bodipy or DiAsp may be used as vital stains.

5. Procedure pursuant to claim 1 characterized because the embryos used in the procedure express the green fluorescent protein gen (Green Fluorescence Protein, GFP), so that the primordium migration can be directly observed under fluorescent lighting and using a dissection loupe.

6. Procedure pursuant to claim 5 characterized because the green fluorescent protein gen (Green Fluorescence Protein, GFP), expresses itself under the control of a specific primordium promoter.

7. Procedure pursuant to claim 6 characterized because the specific primordium promoter is claudinB.

8. The procedure pursuant to claim 1 characterized because it permits the selection of molecules possessing an activity potentially useful in the preparation of pharmaceutical compositions to treat diseases associated with cellular migration defects, such as psoriasis, eczemas; Crohn's disease, colitis; multiple sclerosis, Alzheimer's disease, rheumatoid arthritis, arteriosclerosis, juvenile diabetes, metastasis, immunodeficiency, autoimmune diseases, etc.

9. The procedure pursuant to claim 1 characterized because the molecules to be tested are isolated molecules.

10. The procedure pursuant to claim 1 characterized because the molecules to be tested are contained in cellular extracts.

11. The procedure pursuant to claim 1 characterized because the molecules to be tested may be simultaneously analyzed, be it in diverse concentrations and/or in combinations with different molecules.

12. The procedure pursuant to claim 1 characterized because the embryos are deposited in a small cup (in a tray with 96 small cups) containing a medium that assures the embryos' survival.

13. Isolated molecule, useful in the preparation of pharmaceutical compositions to treat diseases associated with cellular migration characterized because said molecule possesses activity to significantly influence the lateral tine primordium migration in zebra fish embryos (Danio rerio), be it preventing, delaying, accelerating or making their movement erratic and where this activity shown by said molecule has been exclusively detected by the method in claim 1.

14. The molecule pursuant to claim 13 characterized because said molecule is useful in the preparation of pharmaceutical compositions to treat diseases associated with cellular migration defects, such as psoriasis, eczemas; Crohn's disease, colitis; multiple sclerosis, Alzheimer's disease, rheumatoid arthritis, arteriosclerosis, juvenile diabetes, metastasis, immunodeficiency, autoimmune diseases, etc.

15. Use of an isolated molecule in the preparation of pharmaceutical compositions characterized because said molecule is useful in the preparation of pharmaceutical compositions to treat diseases associated with cellular migration defects, such as psoriasis, eczemas; Crohn's disease, colitis; multiple sclerosis, Alzheimer's disease, rheumatoid arthritis, arteriosclerosis, juvenile diabetes, metastasis, immunodeficiency, autoimmune diseases, etc., and where the usefulness of said molecule to treat said diseases, has been exclusively detected by the method in claim 1.
